(19) United States
(12) Patent Application Publication
(10) Pub. No.: US 2025/0295303 A1
(43) Pub. Date: Sep. 25, 2025

(54) ADJUSTABLE CHIN REST APPARATUS FOR VISUAL FIELD SYSTEM

(71) Applicant: Mayo Foundation for Medical Education and Research, Rochester, MN (US)

(72) Inventors: Cheryl L. Khanna, Austin, MN (US); Jonathan M. Morris, Rochester, MN (US); Adam J. Wentworth, Rochester, MN (US); Seyedmostafa Sadegh Mousavi, Rochester, MN (US)

(21) Appl. No.: 18/863,635

(22) PCT Filed: May 10, 2023
(86) PCT No.: PCT/US2023/021692

§ 371 Date: Nov. 6, 2024

Related U.S. Application Data
(60) Provisional application No. 63/340,097, filed on May 10, 2022.

(51) Int. Cl. A61B 3/00; A61B 3/024

(52) U.S. Cl. CPC A61B 3/0083; A61B 3/024

(57) ABSTRACT

Described herein is a chin rest apparatus for a visual field system. The chin rest apparatus can include a base and at least one chin rest insert moveably coupled to the base and configured to be adjusted to one of a plurality of head turn angles and one of a plurality of head tilt angles. The plurality of head turn angles and plurality of head tilt angles can be associated with obtaining a visual field of a subject in the visual field system.

### CROSS-REFERENCE TO RELATED APPLICATIONS

This application is based on, claims priority to, and incorporates herein by reference in its entirety U.S. Ser. No. 63/340,097 filed May 10, 2022 and entitled “Adjustable Chin Rest Apparatus for Visual Field System.”

### STATEMENT REGARDING FEDERALLY SPONSORED RESEARCH

N/A

### BACKGROUND

Certain ocular disorders, such as glaucoma, retinitis pigmentosa, optic neuropathies due to injuries, or toxicity from medications (e.g., corticosteroids, antibiotics, antineoplastic and antiarrhythmics), result in peripheral visual defects. Proper assessment of peripheral visual defects has broad implications across medical specialties, Ideally, an individual's complete visual field would be performed from the central to far periphery in a single field for diseases affecting the visual field to allow accurate assessment of disease severity and progression.

The visual field is essentially the area of space that can be seen at the same time when focusing at one single target. Thereby, the visual field test is the portion of space in which light with different color and size are visible along with fixation of gaze in one direction. The visual field test can consist of two parts of centra and peripheral vision, which includes the inner 30 degrees of vision as the central vision, and 100 degrees laterally, 60 degrees medially, 60 degrees upward, and 75 degrees downward as the peripheral vision. Automated visual field testing may be performed with a visual field machine, which may be called a perimeter. In perimetry, the patient is asked to put their chin on a chin rest area of the machine and doing the whole process of the visual field test while gazing at the target. In the primary or standard position, the vertical axis of the head is vertical to the visual field and tangent to the vertical axis of the machine.

Glaucoma is a major cause of irreversible blindness worldwide with significant quality of life implications. As such early detection of glaucoma is critical in controlling visual deterioration and preserving visual function. In glaucoma, loss of retinal ganglion cells leads to loss of peripheral vision. Functional assessment for measuring glaucoma progression includes visual field testing. Visual field can be assessed with 24-2, 30-2, and 60-4 testing patterns, which vary in the degree of deviation from the central axis measured and number of testing points considered. Notably, central vision can be assessed with 24-2 and 30-2 field patterns; however, peripheral vision beyond 30 degrees of the central visual field axis is assessed with a 60-4 threshold.

The central visual field is more commonly assessed in clinical practice for tracking glaucoma progression. This partly stems from wide variability and unclear appropriate thresholds in 60-4 visual field of healthy control subjects, potentially due to differences in point sensitivity and the potential impact of facial structure. Additionally, in moderate to severe cases of glaucoma, peripheral visual field defects accompany central visual field defects. Unfortunately, in early stages of glaucoma, central and peripheral visual field loss may not be correlated; peripheral defects may manifest in the absence of central field defects. In fact, 11-17% of patients with glaucoma may have peripheral visual field defects in the absence of central visual field defects. Detecting visual field defects associated with glaucoma in the peripheral region may enable earlier detection and treatment of the disease.

Various factors can affect the ability to perform a visual field test for a patient as well as the accuracy of a visual field test. For example, patients with head, facial or body deformities may not be able to perform a visual field due to inability to fully position in a visual field machine. Additionally, facial contours (e.g., nose, cheeks, eyebrows, etc.) can impact far peripheral visual field results when utilizing, for example, a 60-4 testing pattern. The impact of facial structure on field defects may complicate identification of pathological peripheral field defects. Specifically, prominent facial structures may obscure areas of the peripheral field which would otherwise be useful in disease monitoring. Both central and peripheral visual defects have independent diagnostic value and impact on quality of life, with peripheral defects increasing fall risk and alterations in balance. Thus, attainment of an accurate visual field and optimizing strategies for distinguishing facial contour-dependent field defects from pathological defects is paramount for detection of ocular disease progression. Visual field defects caused by facial contours of a subject can be altered or changed by turning or tilting the head of the subject in the visual field system.

Thus, there is a need for visual field systems that can accommodate patients with head, facial or body deformation so that a visual field may be performed for such patients to assess peripheral visual defects, disease severity and progression. In addition, there is a need for visual field systems that are configured to allow the positioning of a subject's head at, for example, a head turn angle that is configured to minimize peripheral visual field defects related to facial structures (or contours). Mapping the visual field from mild to severe disease and correcting for individual variation of facial contour is critical to accurately diagnose and follow disease progression.

### SUMMARY OF THE DISCLOSURE

In accordance with an embodiment, a chin rest apparatus for a visual field system includes a base and at least one chin rest insert moveably coupled to the base and configured to be adjusted to one of a plurality of head turn angles and one of a plurality of head tilt angles. The plurality of head turn angles and plurality of head tilt angles can be associated with obtaining a visual field of a subject in the visual field system.

In accordance with another embodiment, a visual field system includes a testing bowl, a forehead rest and a chin rest apparatus. The chin rest apparatus can include a base and at least one chin rest insert moveably coupled to the base and configured to be adjusted to one of a plurality of head turn angles and one of a plurality of head tilt angles. The plurality of head turn angles and plurality of head tilt angles can be associated with obtaining a visual field of a subject.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1A is a perspective view of a chin rest apparatus for a visual field system in accordance with an embodiment of the invention;

FIG. 1B is an exploded perspective view of the chin rest apparatus of FIG. 1A in accordance with an embodiment;

FIGS. 1C-1D are perspective views of a chin rest apparatus for a visual field system in accordance with an embodiment;

FIG. 1E is a side view of the chin rest apparatus of FIGS. 1C-1D in accordance with an embodiment;

FIG. 1F is a perspective view of the chin rest apparatus of FIGS. 1C-1D in accordance with an embodiment;

FIG. 2A shows elements of an example chin rest apparatus before assembly in accordance with an embodiment;

FIG. 2B shows the assembled elements of the example chin rest apparatus of FIG. 2A in accordance with an embodiment;

FIG. 3A shows a visual field system including a conventional chin rest;

FIG. 3B, shows a visual field system including the chin rest apparatus of FIGS. 1A-2B in accordance with an embodiment;

FIGS. 4A and 4B are perspective views of a chin rest apparatus for a visual field system in accordance with an embodiment;

FIG. 5A illustrates the chin rest assembly of FIGS. 4A and 4B in a visual field system in accordance with an embodiment;

FIG. 5B illustrates a subject positioned in the chin rest apparatus and visual field system of FIG. 5A in accordance with an embodiment;

FIGS. 6A and 6B illustrate an example model for the fabrication of the chin rest apparatus of FIGS. 4A and 4B in accordance with an embodiment;

FIG. 7 is a block diagram of a system for acquiring a visual field of a subject with an adjustable chin rest apparatus and correcting changes to distance and angles from the visual axis to visual field stimulus in accordance with an embodiment;

FIG. 8 is a schematic diagram of a method for calculating a pupil to testing bowl compensation distance in accordance with an embodiment;

FIG. 9A illustrates an example head turn (or rotation) about a vertical axis in accordance with an embodiment;

FIG. 9B illustrates an example series of visual field maps for a subject showing the effect of turning the head of the subject in accordance with an embodiment; and

FIG. 10 is a block diagram of a system for optimizing a head turn of a subject for a visual field test and determining a corrected visual field for the subject in accordance with an embodiment.

### DETAILED DESCRIPTION

The present disclosure describes an adjustable chin rest apparatus for a visual field system. The adjustable chin rest apparatus is configured to allow the positioning of a subject's head at a desired head turn angle and/or head tilt angle within the visual field system. In some embodiments, positioning the subject's head at a particular head turn angle and/or head tilt angle can allow a user with head, facial, or body deformities to be fully positioned in a visual field system in order to obtain a visual field of the subject. In some embodiments, positioning the subject's head at an optimal head turn angle can minimize the visual field defects caused by facial contours of the subject. The present disclosure further describes a method for mathematical correction of the resultant change in distances and angles from the visual axis to the visual field stimulus in the visual field system caused by positioning a subject's head at a particular head turn angle or head tilt angle using the adjustable chin rest apparatus.

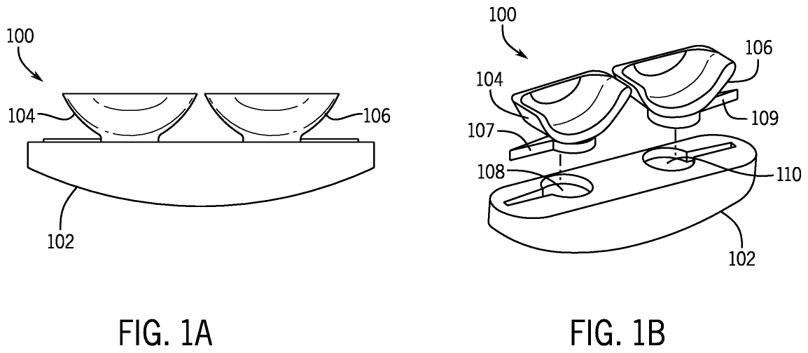

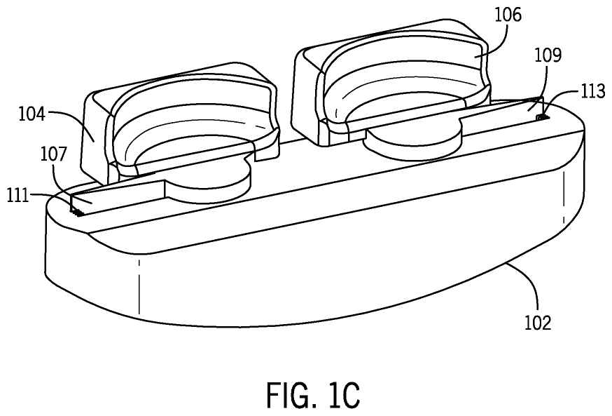

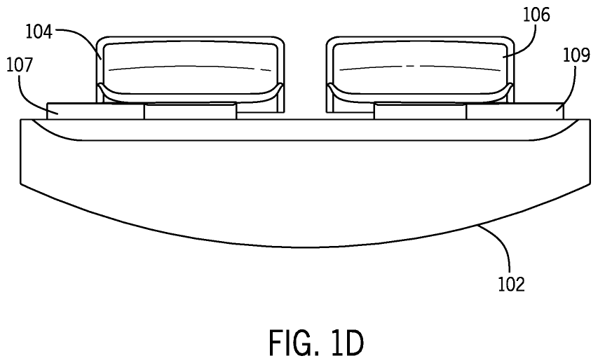

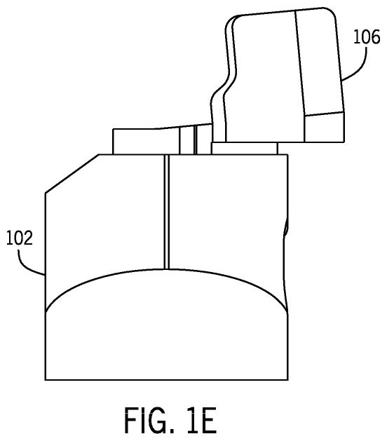

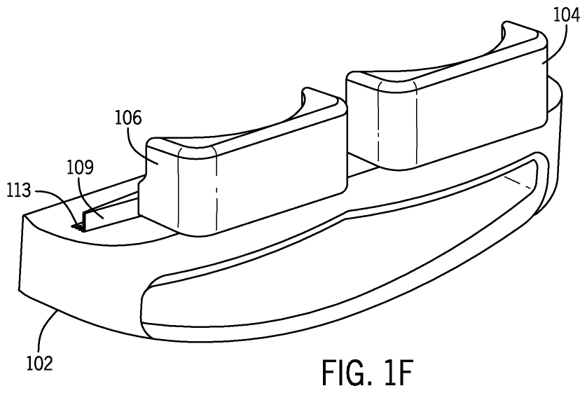

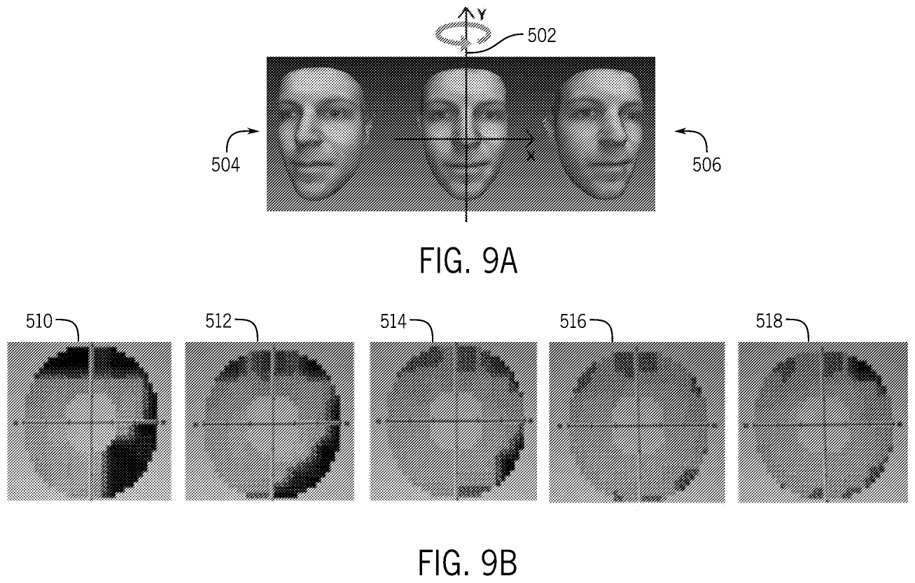

FIG. 1A is a perspective view of a chin rest apparatus for a visual field system in accordance with an embodiment of the invention and FIG. 1B is an exploded perspective view of the chin rest apparatus of FIG. 1A in accordance with an embodiment. As shown in FIGS. 1A and 1B, a chin rest apparatus 100 can include a main body 102. In some embodiments, the main body 102 may be configured to act as an adapter and can allow the chin rest apparatus 100 to be installed (e.g., retrofitted) on an existing visual field system. In some embodiments, the chin rest apparatus 100 may be manufactured as part of a visual field system. The chin rest apparatus 100 also includes a first chin rest insert 104 and a second chin rest insert 106. In some embodiments, the first chin rest insert 104 and the second chin rest insert 106 may be configured to be incrementally tilted to allow the positioning of a subject's head at a desired head tilt angle. As shown in FIG. 1B, the main body 102 may include a first cavity 108 and a second cavity 110 on a top surface 112 of the main body 102. The first cavity 108 may be configured to receive the first chin rest insert 104 and the second cavity 110 may be configured to receive the second chin rest insert 106. In some embodiments, the first cavity 108 and the second cavity 110 are configured to allow the first chin rest insert 104 and the second chin rest insert 106, respectively, to be incrementally rotated to allow the positioning of a subject's head at a desired head turn angle. Accordingly, when placed in the cavity 108, the first chin rest insert 104 may be rotated to provide a desired head turn angle. In addition, when placed in the cavity 110, the second chin rest insert 104 may be rotated to provide a desired head turn angle. For example, in some embodiments, the first chin rest insert 104 and the second chin rest insert 106 may include an insert tab 107 and 109, respectively, that may be rotated into labeled slots 111, 113, respectively (as shown in FIGS. 1C and 1F) in the top surface 112 of the main body 102. In some embodiments, the insert tabs 107 and 109 may have a pointed shape as shown in FIG. 1B. As used herein and as described further below with respect to FIGS. 9A and 9B, the head turn angle may be defined as the amount of turning or rotation of the head of a subject about a vertical axis.

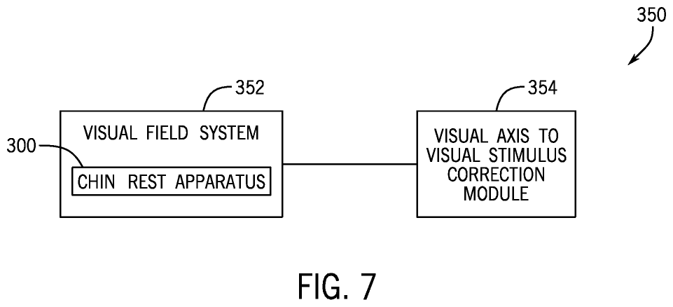

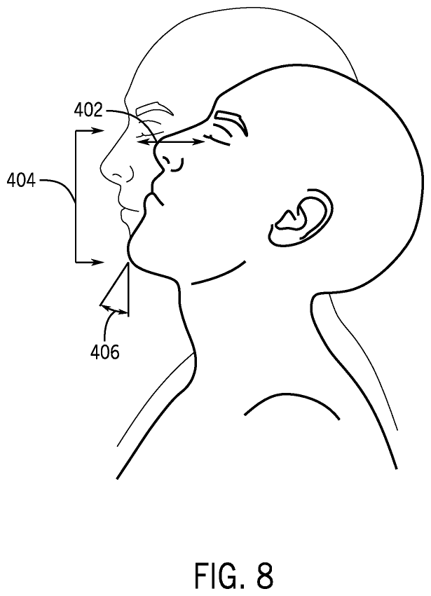

In some embodiments, the first chin rest insert 104 and the second chin rest insert 106 can compensate for changes in pupil to testing bowl distance as a result of tilting and/or turning. For example, the distance of the pupil to the visual field may be kept constant by integration of the resultant offset into the design of the chin rest insert in the visual field system. FIGS. 1C-1F illustrate views of a chin rest apparatus for a visual field system that has been designed to include an offset in accordance with an embodiment. In FIGS. 1C-1F, the first chin rest insert 104 and the second chin rest insert 106 have been designed with a shift towards the testing bowl and the amount of the shift may be based on, for example, the tilt angle of the chin rest insert 104, 106. The determination of offsets or compensation for changes in pupil to testing bowl distance as a result of tilting and/or or turning of the subject's head is discussed further below with respect to FIGS. 7 and 8.

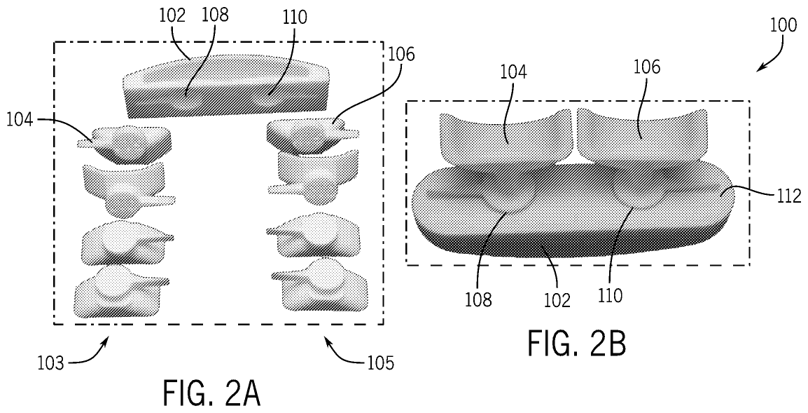

In some embodiments, the first chin rest insert 104 and the second chin rest insert 106 may be labeled with the testing eye (i.e., left or right eye) to which it corresponds and the tilt angle. For example, a plurality of inserts 104, 106 may be provided where each insert corresponds to a combination of a particular testing eye (i.e., left or right) and a particular tilt angle, as discussed further below with respect to FIG. 2A. In some embodiments, an operator can select an insert 104, 106 appropriate for the particular testing eye and the desired tilt angle. Advantageously, in some embodiments, the chin rest apparatus 100 can allow for a visual field to be obtained for subjects with face, head, and body deformities by allowing the patients head to be positioned at a head turn angle and/or head tilt angle that allows the subject's head to be positioned in a visual field system. In addition, in some embodiments, the chin rest apparatus 100 can advantageously be used to position a subject's head at an head turn angle that minimizes the visual field defects caused by the facial contours of the subject which, for example, can correct and optimize the visual field for the subject.

In some embodiments, the chin rest apparatus 100 may be fabricated using three-dimensional (3D) printing technologies such as, for example, powder bed fusion technology. FIG. 2A shows elements of an example chin rest apparatus before assembly in accordance with an embodiment and FIG. 2B shows the assembled elements of the example chin rest apparatus of FIG. 2A in accordance with an embodiment. In FIG. 2A, elements of the chin rest assembly 100, namely, the main body 102 and a set 103 of first 104 chin rest inserts and a set 105 of second 106 chin rest inserts, as shown and may be fabricated using, for example, powder bed fusion 3D printing. As mentioned above, in some embodiments, a plurality of inserts 104, 106 may be provided where each insert corresponds to a combination of a particular testing eye (i.e., left or right) and a particular tilt angle. For example, in FIG. 2A, a set 103 of four labeled first chin rest inserts 104 are shown that correspond to the left eye and tilt the head in increment of 0, 5, 10 and 15 degrees (L0, L5, L10, L15) and a set 105 of four labeled second chin rest inserts 106 are shown that correspond to the right eye and tilt the head in increment of 0, 5, 10 and 15 degrees (R0, R5, R10, R15). In some embodiments, an operator can select an insert 104, 106 appropriate for the particular testing eye and the desired tilt angle. In FIG. 2B, the chin rest assembly 100 assembled using the main body 102 and first 104 and second 106 chin rest inserts is shown. For example, the first chin rest insert 104 and the second chin rest insert 106 are positioned in the first 108 and second 110 cavities on the top surface 112 of the main body 102. When positioned in the first 108 and second 110 cavities, the first 104 and second 106 chin rest inserts may also be used for turning the head of the subject in increments of, for example, 0, 5, 10, and 15 degrees by rotating the insert tab 107 and 109 (e.g., a pointed insert tab), respectively, into labeled slots 111, 113 (shown in FIGS. 1C and 1F) in the top surface 112 of the main body 102.

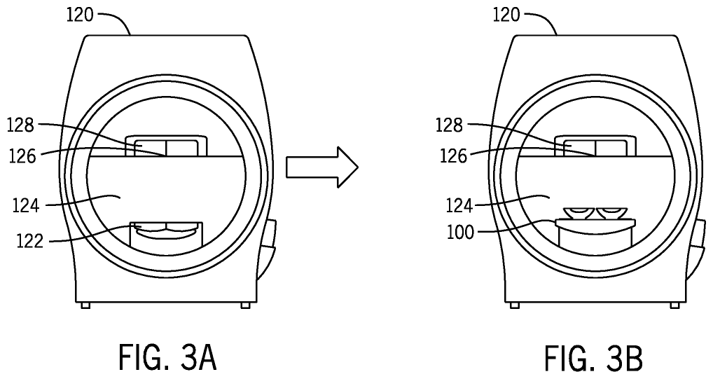

As mentioned above, in some embodiments, the main body 102 of the chin rest apparatus 100 may be configured to act as an adapter and can allow the chin rest apparatus 100 to be installed (e.g., retrofitted) on an existing visual field system. FIG. 3A shows a visual field system including a conventional chin rest and FIG. 3B, shows a visual field system including the chin rest apparatus 100 of, for example, FIGS. 1A-2B in accordance with an embodiment. In FIG. 3A, an example visual field system 120 is shown. The visual field system 120 includes a conventional chin rest 122, a testing bowl 124, a visor handle 126 and a forehead rest 128. In some embodiments, the visual field system 120 may include known mechanisms to move the chin rest 122 to provide vertical (axial displacement) and lateral (sagittal displacement) displacement of a subject's head. Advantageously, in some embodiments, the chin rest apparatus 100 (e.g., as shown in FIGS. 1A-2B) can be retrofitted onto the existing chin rest 122 of the visual field system 120 as shown in FIG. 3B. For example, the chin rest apparatus 100 may be retrofitted onto the existing chin rest using a press fit. In some embodiments, the chin rest apparatus 100 shown in FIG. 3B can be moved to provide vertical and lateral displacement of the subject's head using the existing mechanisms of the visual field system 120. As discussed above, the chin rest apparatus 100 is also advantageously configured to provide a head turn angle and/or a head tilt angle. Advantageously, the chin rest apparatus 100 may be operated within the visual field system 120 without interference between any immobile components of the visual field system 120 and allows normal range of movement (e.g. vertical and lateral displacement) of the existing chin rest on which the chin rest apparatus 100 is fitted. In some embodiments, dynamic stabilization of a subject's forehead could be accomplished by attachment of, for example, foam to the forehead rest 128.

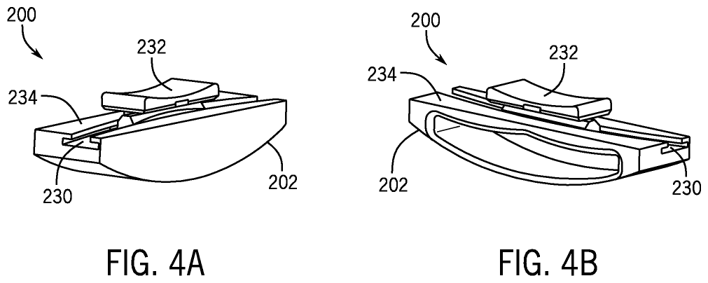

FIGS. 4A and 4B are perspective views of a chin rest apparatus for a visual field system in accordance with an embodiment. As shown in FIGS. 4A and 4B, a chin rest apparatus 200 can include a main body 202. In some embodiments, the main body 202 may be configured to act as an adapter and can allow the chin rest apparatus 200 to be installed (e.g., retrofitted) on an existing visual field system (e.g., the visual field system 120 show in FIG. 3A). For example, the chin rest apparatus 200 may be retrofitted onto an existing chin rest of a visual field system using a press fit. In some embodiments, the chin rest apparatus 200 may be manufactured as part of a visual field system. The chin rest apparatus 200 also includes a groove 230 on a top surface 234 of the main body 202 and a chin rest insert 232. The chin rest insert 232 may be configured to be positioned in the groove 230 and to slide along the path defined by the groove 230. In some embodiments, the groove 230 may define a parabolic path. In some embodiments, the chin rest insert 232 may be slid along the groove 230 to provide and accommodate a desired head turn angle for the subject's head and the chin rest insert 232 may also be configured to be tilted to allow the positioning of a subject's head at a desired head tilt angle. In some embodiments, the groove 230 may define a parabolic path that allows the chin rest insert 232 as it is slid along the parabolic path to accommodate a head turn angle of up to 13.5 degrees in either direction. In some embodiments, the groove 230 and chin rest inserts 232 may be configured to accommodate a head tilt angle up to ±45 degrees and the chin rest insert 232 may be positioned at any angle within this range. Advantageously, in some embodiments, the chin rest apparatus 200 can allow for a visual field to be obtained for subjects with face, head, and body deformities by allowing the patients head to be positioned at a head turn angle and/or head tilt angle that allows the subject's head to be positioned in a visual field system. In addition, in some embodiments, the chin rest apparatus 200 can advantageously be used to position a subject's head at a head turn angle that minimizes the visual field defects caused by the facial contours of the subject which, for example, can correct and optimize the visual field for the subject.

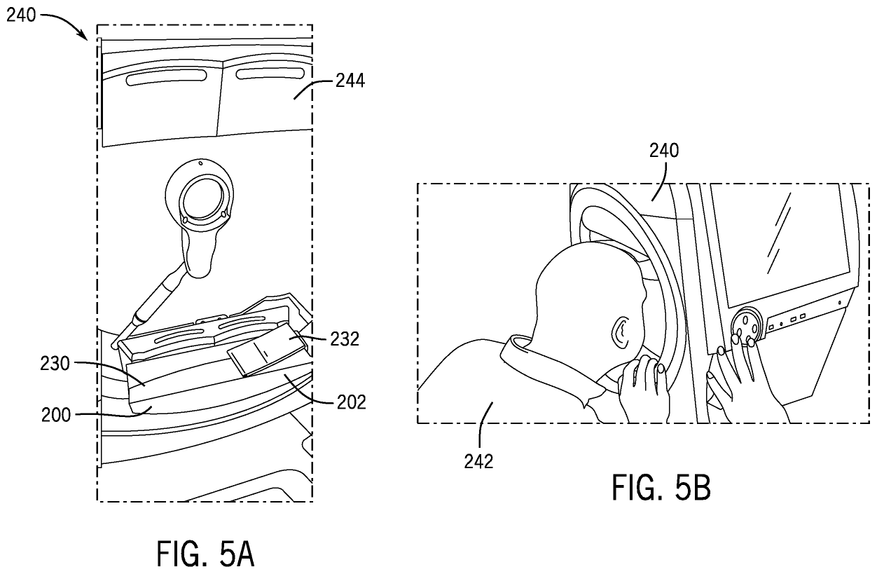

As mentioned above, in some embodiments, the main body 202 of the chin rest apparatus 200 may be configured to act as an adapter and can allow the chin rest apparatus 200 to be installed (e.g., retrofitted) on an existing visual field system. FIG. 5A illustrates the chin rest assembly of FIGS. 4A and 4B in a visual field system in accordance with an embodiment. In FIG. 5A, the main body 202 of a chin rest apparatus 200 has been positioned (e.g., retrofitted) onto a visual field system 240. For example, in some embodiments, the chin rest apparatus 200 can be retrofitted onto the existing chin rest of the visual field system 240. In some embodiments, the visual field system 240 may include known mechanisms to move the chin rest 200 to provide vertical (axial displacement) and lateral (sagittal displacement) displacement of a subject's head. In some embodiments, the chin rest apparatus 200 shown in FIG. 5A can be moved to provide vertical and lateral displacement of the subject's head using the existing mechanisms of the visual field system 240. As discussed above, the chin rest apparatus 100 is also advantageously configured to provide a head turn angle and/or a head tilt angle. Advantageously, the chin rest apparatus 200 may be operated within the visual field system 240 without interference between any immobile components of the visual field system 240 and allows normal range of movement (e.g. vertical and lateral displacement) of the existing chin rest on which the chin rest apparatus 200 is fitted. In some embodiments, dynamic stabilization of a subject's forehead could be accomplished by attachment of, for example, foam to a forehead rest 244 of the visual field system 240.

In FIG. 5A, the chin rest insert 232 is shown located along the groove 230 towards a right side of the visual field system 240 and the chin rest insert 232 is positioned at an angle (i.e., the head turn angle). The angle of the chin rest insert 232 allows a subject's head that is placed in the chin rest insert 232 to be positioned at the same angle in the visual field system 240. FIG. 5B illustrates a subject 242 positioned in the chin rest apparatus 232 and visual field system 240 of FIG. 5A.

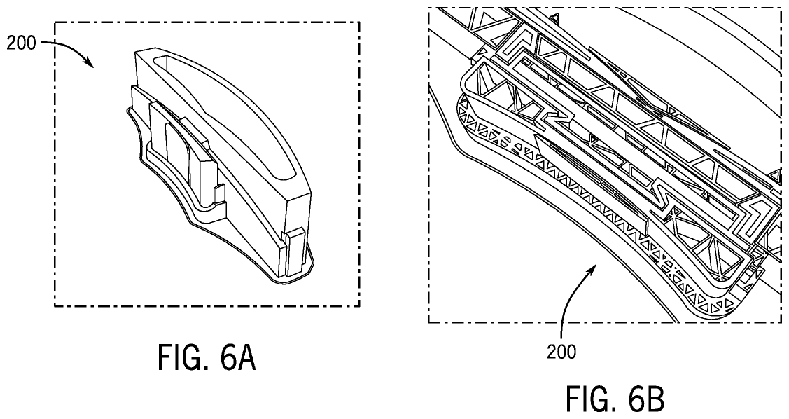

In some embodiments, the chin rest apparatus 200 may be fabricated using three-dimensional (3D) printing technologies such as, for example, powder bed fusion technology. In some embodiments, the chin rest apparatus 200 may advantageously be fabricated as a single 3D printed object utilizing, for example, fused deposition modeling 3D dissolvable support material. FIGS. 6A and 6B illustrate an example model for the fabrication of the chin rest apparatus 200 of FIGS. 4A and 4B in accordance with an embodiment. In particular, FIGS. 6A and 6B illustrate the chin rest apparatus 200 as a single part with embedded moveable components.

In some embodiments, the resultant change in distances and angles from the visual axis to the visual field stimulus in the visual field system caused by positioning a subject's head at a particular head turn angle or head tilt angle using the adjustable chin rest apparatus 100, 200 may be corrected (e.g., using a determined offset). In some embodiments, the distance of the pupil to the visual field may be kept constant by integration of the resultant offset into the design and positioning of the chin rest insert (e.g., chin rest insert 104, 106, 232) in the visual field system. For example, in some embodiments, the position of the chin rest insert may be shifted towards the testing bowl as the head of the subject tilts upward. In some embodiments, the chin rest insert is designed with a shift towards the testing bowl (e.g., as shown in FIGS. 1C-1F). In some embodiments, the lateral and vertical changes as a result of head turn/tilt may be corrected by an electronic adjustment of the position of the existing chin rest of the visual field system on which the chin rest apparatus 100, 200 may be installed as described above. FIG. 7 is a block diagram of a system for acquiring a visual field of a subject with an adjustable chin rest apparatus and correcting changes to distance and angles from the visual axis to visual field stimulus in accordance with an embodiment. The system 350 includes a visual field system 352, and a visual axis to visual stimulus correction module 354. The visual field system 352 includes a chin rest apparatus 300 such as, for example, the chin rest apparatus 100 discussed above with respect FIGS. 1A-3B or the chin rest apparatus 200 discussed above with respect to FIGS. 4A-6B. In some embodiments, elements of system 350 may be implemented in the same device. In other embodiments, various elements are implemented in different locations or devices and may be in signal communication via wired or wireless connections. For example, the visual axis to visual stimulus correction module 354 may be implemented as part of the visual field system 352 (e.g., using a processor), or may be implemented on a processor of a separate computer system.

In some embodiments, the processor on which the visual axis to visual stimulus correction module 354 is implemented may be included in any general-purpose computing system or device, such as a personal computer, workstation, cellular phone, smartphone, laptop, tablet, or the like. The processor may include any suitable hardware and components designed or capable of carrying out a variety of processing and control tasks, including steps for determining corrections to distance and angles from the visual axis to visual field stimulus, determining a corrected visual field of a subject, or determining an optimized head turn angle for determining a visual field of a subject. For example, the processor may include a programmable processor or combination of programmable processors, such as central processing units (CPUs), graphics processing units (GPUs), and the like. In some implementations, the processor may be configured to execute instructions stored in a non-transitory computer readable-media. In this regard, the processor may be any device or system designed to integrate a variety of software, hardware, capabilities and functionalities. Alternatively, and by way of particular configurations and programming, the processor may be a special-purpose system or device. For instance, such special-purpose system or device may include one or more dedicated processing units or modules that may be configured (e.g., hardwired, or pre-programmed) to carry out steps, in accordance with aspects of the present disclosure.

The visual field system 352 may be any visual field system that is configured to perform different types of visual field tests that each measure various degrees of peripheral vision including, but not limited to, a 10 degree visual field (e.g., 10-2), a 30 degree visual field (e.g., 30-2) and a 60 degree visual field (e.g., 60-4), or a combination of fields, including central, mid peripheral, and/or far peripheral. The visual field tests may be performed on a subject by the visual field system 352 using known methods. The acquired visual field may be for a right eye of the subject or a left eye of the subject. The visual field of a subject acquired using the visual field system 352 may be stored in data storage (or memory), for example, data storage of the visual field system 352, or other computer system. As discussed above, the chin rest apparatus 300 may be used to position a subject's head at a desired head turn angle and/or head tilt angle for acquisition of the visual field using the visual field system 352.

The visual axis to visual stimulus correction module 354 can be configured to determine a correction for the resultant changes in distances and angles from the visual axis to the visual field stimulus in the visual field system 352 caused by positioning a subject's head at a particular head turn angle or head tilt angle using the adjustable chin rest apparatus 300. For example, as mentioned above, a pupil to testing bowl compensation distance may be calculated and the compensation distance or offset used to position the chin rest insert (or chin rest apparatus) in the visual field system to correct for the changes in the distance and angle from the visual axis to the visual field stimulus. FIG. 8 is a schematic diagram of a method for calculating a pupil to testing bowl compensation distance in accordance with an embodiment. In some embodiments, a pupil to testing bowl compensation distance 402 may be calculated based on a Sellion-Menton length 8-4 and a tilt angle 406 of the subject's head. In some embodiments, data such as, for example, bipupil breadth and Sellion-Menton length may be used to obtain average lengths and to calculate pupil to testing bowl, vertical, and lateral offset distances as a result of a head turn and tilt. The bipupil breadth may be defined as the bilateral distance between the right and left pupil centers of the eyes when looking straight ahead and the Sellion-Menton length may be defined as the midsagittal distance between the Sellion and Mention landmarks with the teeth in occlusion. In an example, data regarding bipupil breadth and Sellion-Mention length may be provided from the U.S. Government document “Head and Face Anthropometry of Adult U.S. Citizens (1993).,” herein incorporated by reference in its entirety.

In some embodiments, the offset distances may be calculated using the chin as the point of rotation, vertical distance between the chin and pupil as anthropomorphic average of the Sellion-Menton distance, and horizontal distance between the chin and pupil as half of the anthropomorphic average of the interpupillary distance. In an example, the Sellion-Menton average=116 mm±7 mm, average of 195 male and 172 female and the interpupillary distance average=60 mm±2 mm, average of 136 male and 102 female. In some embodiments, the head tilt offset distance may be determined using the following equations:

$$Pupil to testing bowl distance change = Sellion - Menton avg * sin ( tilt angle ) \qquad ( 1 )$$

$$Vertical Shift = Sellion - Menton Avg ( chin to nose bridge ) - \sqrt{{( Sellion - Menton Avg )}^{2} - {( pupil to testing bowl distance change )}^{2}} \qquad ( 2 )$$

In some embodiments, the head turn offset distance may be determined using the following equations:

$$Pupil to testing bowl distance change = Interpupillary distance * sin ( turn angle ) / sin ( 90 ) \qquad ( 3 )$$

$$Lateral Shift = Interpupillary distance - ( Interpupillary distance * sin ( 90 - turn angle ) / sin ( turn angle ) ) \qquad ( 4 )$$

An example of calculated tilt and turn offset distances (or compensation) are shown below in Table 1.

TABLE 1
Independent head tilt and turn offsets for degrees 1-15.

| | | | | |
|---|---|---|---|---|
| forward/back | vert shift | lateral shift | tilt degree | turn degree |

HEAD TILT OFFSET CALCULATIONS
as head tilts up, chin must move closer to screen, and up

| | | | | |
|---|---|---|---|---|
| −2.024 | 0.018 | 0 | 1 | 0 |
| −4.048 | 0.071 | 0 | 2 | 0 |
| −6.071 | 0.159 | 0 | 3 | 0 |
| −8.092 | 0.283 | 0 | 4 | 0 |
| −10.110 | 0.441 | 0 | 5 | 0 |
| −12.125 | 0.635 | 0 | 6 | 0 |
| −14.137 | 0.865 | 0 | 7 | 0 |
| −16.144 | 1.129 | 0 | 8 | 0 |
| −18.146 | 1.428 | 0 | 9 | 0 |
| −20.143 | 1.762 | 0 | 10 | 0 |
| −22.134 | 2.131 | 0 | 11 | 0 |
| −24.118 | 2.535 | 0 | 12 | 0 |
| −26.094 | 2.973 | 0 | 13 | 0 |
| −28.063 | 3.446 | 0 | 14 | 0 |
| −30.023 | 3.953 | 0 | 15 | 0 |

HEAD TURN OFFSET CALCULATIONS
as head turns away from screen, chin must move away from screen,
and laterally opposite of the turning direction

| | | | | |
|---|---|---|---|---|
| 0.524 | 0 | 0.005 | 0 | 1 |
| 1.047 | 0 | 0.018 | 0 | 2 |
| 1.570 | 0 | 0.041 | 0 | 3 |
| 2.093 | 0 | 0.073 | 0 | 4 |
| 2.615 | 0 | 0.114 | 0 | 5 |
| 3.136 | 0 | 0.164 | 0 | 6 |
| 3.656 | 0 | 0.224 | 0 | 7 |
| 4.175 | 0 | 0.292 | 0 | 8 |
| 4.693 | 0 | 0.369 | 0 | 9 |
| 5.209 | 0 | 0.456 | 0 | 10 |
| 5.724 | 0 | 0.551 | 0 | 11 |
| 6.237 | 0 | 0.656 | 0 | 12 |
| 6.749 | 0 | 0.769 | 0 | 13 |
| 7.258 | 0 | 0.891 | 0 | 14 |
| 7.765 | 0 | 1.022 | 0 | 15 |

An example set of calculated offset distances as a function of combinations of tilt and turn angles are shown below in Table 2.

TABLE 2
Calculated compensation values in mm for respective tilt and turn angles.
Chin rest offsets as a function of combinations of tilt and turn [mm]

| | Tilt (up/down) [degrees] | | | | | | | |
|---|---|---|---|---|---|---|---|---|
| | 0 | | 5 | | 10 | | 15 | |
| Turn left-right | closer | lateral | closer | raise | closer | raise | closer | raise |
| 0 | 0 | 0 | −10.11 | 0.44 | −20.14 | 1.76 | −30.02 | 3.95 |
| 5 | 2.615 | 0.114 | −7.495 | 0.44 | −17.525 | 1.76 | −27.405 | 3.95 |
| 10 | 5.209 | 0.455 | −4.901 | 0.44 | −14.931 | 1.76 | −24.811 | 3.95 |
| 15 | 7.765 | 1.022 | −2.345 | 0.44 | −12.375 | 1.76 | −22.255 | 3.95 |

As mentioned above, the calculated compensation values may be used to modify the design and positioning of the chin rest insert, for example, shifting the head position closer to the screen as head tilts upward. In some embodiments, the compensation may be made only for head tilt, as this results in the most dramatic distance changes. In some embodiments, additional chin rest inserts may be designed to compensate for pupil to testing bowl distance changes as a function of head turning. It should be noted as not all subjects match the average Sellion-Menton and interpupillary distances, and advantageously these integrated offsets may be a practical compromise. In some embodiments, the lateral and vertical changes as a result of head turn/tilt may be corrected by an electronic adjustment of the position of the existing chin rest of the visual field system on which the chin rest apparatus 100, 200 may be installed as described above.

As mentioned above, the described chin rest apparatus 100, 200 may be used to position a subject's head at a head turn angle that changes or alters (e.g., minimizes) the visual field defects caused by the facial contours of the subject. In particular, visual field defects caused by the facial contours of the subject may be altered, for example, by turning the subject's head relative to a vertical axis towards (i.e. temporally) or away from (i.e., nasally) the eye being tested using a visual field system. In some embodiments, an optimal head position (e.g., turning the head to an optimal head turn angle) in the visual field system may be used to maximize the visual field of the subject. The amount of head turn to maximize the visual field for each individual can be different. FIG. 9A illustrates an example head turn (or rotation) about a vertical axis in accordance with an embodiment. In FIG. 9A, a first head turn 504 is shown about a vertical axis 502 to the right and a second head turn 506 is shown to the left. As mentioned above, as used herein, the head turn angle may be defined as the amount of turning or rotation of the head about the vertical axis 502. The head turn may be either toward (i.e., temporally) or away from (i.e., nasally) the tested eye.

FIG. 9B illustrates an example series of visual field maps for a subject showing the effect of turning the head of the subject in accordance with an embodiment. In the example of FIG. 6B, the visual field maps 510, 512, 514, 516 and 518 are 60-4 visual fields. In FIG. 9B, each visual field map 510, 512, 514, 516 and 518 represents a different head position or head turn angle for the subject. In this example, visual field map 510 represents a 25-30° head turn toward (i.e., temporally) the eye being tested. Visual field map 512 represents a 10-15° head turn toward the eye being tested. Visual field map 514 represents the head in a primary position (i.e., no head turn). Visual field map 516 represents a 10-15° head turn away from (i.e., nasally) the tested eye. Visual field map 518 represents a 25-30° head turn away from the tested eye. In the example shown in FIG. 9B, the visual field defects decreased when the head was turned away from the tested eye and the visual field defects increased when the head was turned towards the tested eye. By turning the head about the vertical axis 502 (shown in FIG. 9A) in the opposite direction from the tested eye, the tested eye is abducted when fixating on the central target, and therefore the influence of the nose on the nasal visual field is minimized. Accordingly, the subject has a more accurate visual field when the head was turned away from the tested eye. As mentioned, the amount of head turn to maximize the visual field for each individual is different.

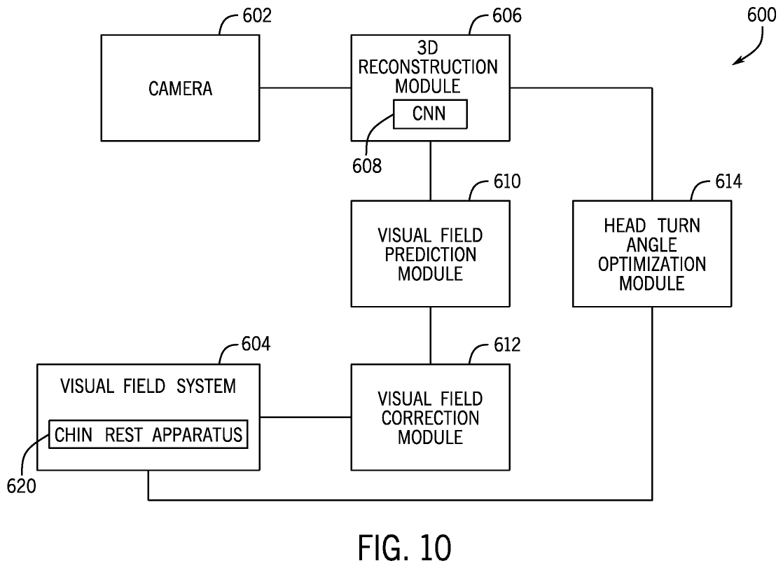

FIG. 10 is a block diagram of a system for optimizing a head turn of a subject for a visual field test and determining a corrected visual field for the subject in accordance with an embodiment. The system 600 includes a camera 602, a visual field system 604 that includes an adjustable chin rest apparatus 620, a three-dimensional (3D) reconstruction module 606 that includes a convolutional neural network (CNN) 608, a visual field prediction module 610, a visual field correction module 612, and a head turn angle optimization module 614. In various embodiments, elements of system 600 may be implemented in the same device. In other embodiments, various elements are implemented in different locations or devices and may be in signal communication via wired or wireless connections. For example, the 3D reconstruction module 606, visual field prediction module 610 and visual field correction module 612, and head turn optimization module 614 may be implemented as part of the visual field system 604 (e.g., using a processor), or may be implemented on a processor of a separate computer system.

As mentioned, the 3D reconstruction module 606, visual field prediction module 610 and visual field correction module 612, and head turn optimization module 614 may be implemented on a processor. In some implementations, the processor may be included in any general-purpose computing system or device, such as a personal computer, workstation, cellular phone, smartphone, laptop, tablet, or the like. The processor may include any suitable hardware and components designed or capable of carrying out a variety of processing and control tasks, including steps for determining a corrected visual field of a subject or determining an optimized head turn angle for determining a visual field of a subject. For example, the processor may include a programmable processor or combination of programmable processors, such as central processing units (CPUs), graphics processing units (GPUs), and the like. In some implementations, the processor may be configured to execute instructions stored in a non-transitory computer readable-media. In this regard, the processor may be any device or system designed to integrate a variety of software, hardware, capabilities and functionalities. Alternatively, and by way of particular configurations and programming, the processor may be a special-purpose system or device. For instance, such special-purpose system or device may include one or more dedicated processing units or modules that may be configured (e.g., hardwired, or pre-programmed) to carry out steps, in accordance with aspects of the present disclosure.

The camera 602 may be any standard camera known in the art that may be used to acquire a two-dimensional (2D) image (i.e., a photograph) of a subject. In particular, the camera 602 may be used to acquire one or more 2D images of a face of a subject. In an embodiment, the 2D image is an RGB image. The 2D image(s) of the face of the subject acquired by the camera 602 may be stored in data storage (or memory), for example, data storage of the camera 602, the visual field system 604, or other computer system. In some embodiments, the 2D images of the face of the subject may be stored as high-resolution JPEG images.

The visual field system 604 may be any visual field system that is configured to perform different types of visual field tests that each measure various degrees of peripheral vision including, but not limited to, a 10 degree visual field (e.g., 10-2), a 30 degree visual field (e.g., 30-2) and a 60 degree visual field (e.g., 60-4), or a combination of fields, including central, mid peripheral, and/or far peripheral. The visual field tests may be performed on a subject by the visual field system 604 using known methods. The acquired visual field may be for a right eye of the subject or a left eye of the subject. The visual field of a subject acquired using the visual field system 604 may be stored in data storage (or memory), for example, data storage of the visual field system 604, or other computer system. The visual field system 604 can include a chin rest apparatus 620 such as, for example, the chin rest apparatus 100 discussed above with respect FIGS. 1A-3B or chin rest apparatus 200 discussed above with respect to FIGS. 4A-6B. As discussed above, the chin rest apparatus 620 may be used to position a subject's head at a desired head turn angle and/or head tilt angle for acquisition of the visual field using the visual field system 604. In some embodiments, the desired head turn angle may be an optimal head turn angle determined using the head turn angle optimization module 614.

The 3D reconstruction module 606 is configured to receive one or more 2D images (i.e., photographs) of a face of a subject from the camera 602. The 2D image of the face of a subject may be, for example, transmitted from the camera 602 via a communication link or retrieved from data storage (or memory). The 3D reconstruction module 606 includes a convolutional neural network (CNN) 608 that is configured to generate a 3D reconstruction of the face of the subject using the 2D image (or images) of the face of the subject. The CNN 608 may be trained using known or developed methods. The 3D reconstruction of the face of the subject may be stored in data storage (or memory), for example, data storage of the visual field system 604, or other computer system. The 3D reconstruction of the face of the subject generated by the 3D reconstruction module 606 may be provided to the head turn angle optimization module 614 coupled to the 3D reconstruction module 606.

The head turn angle optimization module 614 may be configured to determine an optimal head turn angle for a subject based on the 3D reconstruction of the face of a subject. In some embodiments, a plurality of angles theta (θ) for the 360° surrounding a visual axis on the 3D reconstruction may be calculated. Accordingly, angle theta (θ) is calculated for all points circumferential to a visual axis on the 3D reconstruction of the face. In an embodiment, the angles θ along with the coordinates of the points may be stored in a data structure. The smallest (or minimum) angle theta may be identified from the calculated angles theta. An optimal head turn angle, K, may be determined based on the smallest angle θ. As mentioned above, the head turn angle may be defined as the amount of turning or rotation of the head about a vertical axis. The head turn may be either toward (i.e., temporally) or away from (i.e., nasally) the tested eye. In some embodiments, the optimal head turn angle, K, is determined by subtracting the smallest angle θ from a preset angle (e.g., 60 degrees).

In some embodiments, the optimal head turn angle determined by the head turn angle optimization module 614 may be configured to maximize the visual field of the subject acquired, for example, using the visual field system 604. In an embodiment, by positioning the subject's head in the visual field system 604 at the optimal head turn angle when acquiring a visual field, the visual field defects caused by facial contour can be minimized. The optimal head turn angle may be provided to the visual field system 604. In some embodiments, an operator may then position the subject's head at the optimal angle in the visual field system 604 and perform a visual field test using the visual field system to acquire a visual field of the subject. In some embodiments, the subject's head may be positioned in the visual field system 604 at the optimal field angle using the chin rest apparatus 620. By adjusting the subject's head with, for example, turning and tilting, the facial anatomy can be overcome and the maximal far peripheral field can be mapped. Once a visual field has been acquired (e.g., using visual field system 604) with the subject's head at the optimal position determined by the head turn angle optimization module 614, the acquired visual field at the optimal head position may be corrected to eliminate any residual facial contour visual field defects, for example, using the visual field prediction module 610 and the visual field correction module 612.

The 3D reconstruction of the face of the subject generated by the 3D reconstruction module 606 may also be provided to the visual field prediction module 610 coupled to the 3D reconstruction module 606. The visual field prediction module 610 is configured to generate a predicted visual field of the subject indicating predicted visual field defects from facial contours (or structures) of the subject such as, for example, nose, cheeks, eyebrows, etc. The facial contour of a subject may be influenced by factors such as age, race and gender. The predicted visual field is generated using the 3D reconstruction of the face of the subject. In some embodiments, the predicted visual field is a 60-4 visual field. The predicted visual field may be for a right eye of the subject or a left eye of the subject. The predicted visual field for the subject may be stored in data storage (or memory), for example, data storage of the visual field system 604, or other computer system.

The visual field correction module 612 is coupled to the visual field prediction module 610. The predicted visual field for the subject may be provided to the visual field correction module 612. In addition, the visual field correction module 612 may be configured to receive an acquired visual field for the subject from the visual field system 604 (e.g., an visual field acquired using an optimal head turn angle determined by the head turn angle optimization module 114). The acquired visual field for the subject may be, for example, transmitted from the visual field system 604 via a communication link or retrieved from data storage (or memory). In an embodiment, the acquired visual field may be a central, mid peripheral, far peripheral, or combination visual field. The visual field correction module 612 may be configured to generate a corrected visual field for the subject. In some embodiments, the corrected visual field may be generated by subtracting the predicted visual field for the subject from the acquired visual field for the subject. In some embodiments, the corrected visual field may be generated using a numerical correction method. Accordingly, the visual field defects from facial contours can be removed from the acquired visual field. In some embodiments, the acquired visual field at the optimal head position may be corrected to eliminate any residual facial contour visual field defects. The corrected visual field may be for a right eye of the subject or a left eye of the subject. The corrected visual field for the subject may be stored in data storage (or memory), for example, data storage of the visual field system 604, or other computer system. In an embodiment, the corrected visual field for the subject may be displayed on a display, for example, a display of visual field system 604, or other computer system.

The present disclosure has described one or more preferred embodiments, and it should be appreciated that many equivalents, alternatives, variations, and modifications, aside from those expressly stated, are possible and within the scope of the invention.

1. A chin rest apparatus for a visual field system comprising:
a base;
at least one chin rest insert moveably coupled to the base and configured to be adjusted to one of a plurality of head turn angles and one of a plurality of head tilt angles, wherein the plurality of head turn angles and plurality of head tilt angles are associated with obtaining a visual field of a subject in the visual field system.

2. The chin rest apparatus according to claim 1, wherein the at least one chin insert comprises a first chin rest insert and a second chin rest insert.

3. The chin rest apparatus according to claim 2, wherein the base comprises a top surface, a first cavity on the top surface that is configured to receive the first chin rest insert, and a second cavity on the top surface that is configured to receive the second chin rest insert.

4. The chin rest apparatus according to claim 3, wherein the first chin rest insert is associated with a left eye of the subject and the second chin rest insert is associated with a right eye of the subject.

5. The chin rest apparatus according to claim 3, wherein the first chin rest insert is configured to be rotated in the first cavity and the second chin rest insert is configured to be rotated in the second cavity.

6. The chin rest apparatus according to claim 1, wherein the base comprises a top surface and a groove disposed in the top surface.

7. The chin rest apparatus according to claim 6, wherein the groove defines a parabolic path.

8. The chin rest apparatus according to claim 6, wherein the at least one chin insert comprises a single chin insert configured to be positioned in the groove and moved along the groove.

9. The chin rest apparatus according to claim 8, wherein movement of the chin rest insert along the groove changes a position of the chin rest insert resulting in adjustment of the chin rest insert to a desired head turn angle.

10. The chin rest apparatus according to claim 8, wherein the base and chin rest insert are fabricated as a single part.

11. A visual field system comprising:
a testing bowl;
a forehead rest, and
a chin rest apparatus comprising:
a base;
at least one chin rest insert moveably coupled to the base and configured to be adjusted to one of a plurality of head turn angles and one of a plurality of head tilt angles, wherein the plurality of head turn angles and plurality of head tilt angles are associated with obtaining a visual field of a subject.

12. The visual field system according to claim 11, wherein the at least one chin insert comprises a first chin rest insert and a second chin rest insert.

13. The visual field system according to claim 12, wherein the base comprises a top surface, a first cavity on the top surface that is configured to receive the first chin rest insert and a second cavity on the top surface that is configured to receive the second chin rest insert.

14. The visual field system according to claim 12, wherein the first chin rest insert is configured to be rotated in the first cavity and the second chin rest insert is configured to be rotated in the second cavity.

15. The visual field system according to claim 11, wherein the base comprises a top surface and a groove disposed in the top surface.

16. The visual field system according to claim 15, wherein the at least one chin insert comprises a single chin insert configured to be positioned in the groove and moved along the groove.

17. The visual field system according to claim 16, wherein movement of the chin rest insert along the groove changes a position of the chin rest insert resulting in adjustment of the chin rest insert to a desired head turn angle.
